(19) United States
(12) Patent Application Publication
(10) Pub. No.: US 2017/0231706 A1
(43) Pub. Date: Aug. 17, 2017

(54) ILLUMINATED SURGICAL BARRIER

(71) Applicant: Invuity, Inc., San Francisco, CA (US)

(72) Inventor: Alex Vayser, Mission Viejo, CA (US)

(73) Assignee: Invuity, Inc., San Francisco, CA (US)

(21) Appl. No.: 15/435,818

(22) Filed: Feb. 17, 2017

Related U.S. Application Data
(60) Provisional application No. 62/296,509, filed on Feb. 17, 2016.

(51) Int. Cl. A61B 46/10; A61B 90/00; A61B 90/30; A61B 5/00; A61B 90/20

(52) U.S. Cl. CPC A61B 46/10; A61B 5/0071; A61B 90/20; A61B 90/30; A61B 90/361; A61B 2090/306; A61B 2090/309

(57) ABSTRACT

A surgical barrier or cover for covering a surgical instrument comprises an elongate bag having an inner surface, an outer surface, and a wall therebetween. The inner surface defines a cavity that is configured to receive the surgical instrument. A plurality of optical fibers or illumination elements is coupled to the elongate bag. The plurality of optical fibers or illumination elements are configured to deliver light from a light source to the distal end of the elongate bag.

### CROSS-REFERENCE TO RELATED APPLICATIONS

The present application is a non-provisional of, and claims the benefit of US Provisional Patent Application No. 62/296,509 (Attorney Docket No. 4556-750.101) filed Feb. 17, 2016; the entire contents of which are incorporated herein by reference.

### BACKGROUND OF THE INVENTION

1. Field of the Invention

The present application generally relates to medical devices, systems and methods, and more particularly relates to barriers that are preferably sterile, such as drapes or covers or other items used during medical procedures to keep a tool, instrument, device or other work piece segregated from a sterile field during a medical or other procedure.

Equipment used in the operating theatre must be cleaned, sanitized or sterilized to prevent infection or contamination of the surgical field or work area. This can be a time consuming and costly procedure. Some equipment may not withstand the chemicals or temperatures used to perform the cleaning, sanitization or sterilization. Therefore, clean and/or sterile barriers such as, but not limited to drapes are often used to cover equipment or other items used during the procedure. Drapes may be sheets of material with or without fenestrations that are wrapped around the equipment, or the drapes may be covers or bags into which the equipment may be inserted and then the end of the cover may be closed. Often the drapes or covers are fabricated from a fabric, non-woven fabric, or a polymer.

Drapes may be used to cover optical equipment such as lighting, microscopes or cameras. While commercially available covers provide a sterile barrier and prevent contamination of the equipment, existing covers do not always interface with optical equipment so that lighting or image quality is optimal. Similarly, the barrier may not be compatible with sensor instruments since the output from the sensor or the input to the sensor from the surgical field may be distorted by the barrier. Therefore, it would be desirable to provide improved drapes, covers or barriers that can be used with optical equipment, sensors, or other items during a medical procedure or other procedure.

At least some of these challenges will be overcome by the exemplary embodiments disclosed herein.

2. Description of the Background Art

U.S. Pat. No. 9,055,962 describes use of a sterile barrier.

### SUMMARY OF THE INVENTION

The present invention generally relates to medical devices, systems and methods, and more particularly relates to barriers either sterile or non-sterile, such as drapes or covers used during medical or other procedures. One of skill in the art will appreciate that the drapes disclosed herein are not limited to use during medical procedures and they can be used in any other application and with any other instrument, device or equipment. One of skill in the art will also appreciate that sterile barriers are not limited to drapes.

In a first aspect, a barrier such as a surgical drape is preferably sterile and used for covering a surgical instrument, and comprises an elongate bag having an inner surface, an outer surface, and a wall therebetween. The inner surface defines a cavity that is configured to receive the surgical instrument or other device which may remain non-sterile. The surgical drape also comprises a plurality of optical fibers or illumination elements coupled to the elongate bag. The plurality of optical fibers or illumination elements are configured to deliver light from a light source to the distal end of the elongate bag, on in the case where the illumination elements are the illumination source, the illumination elements deliver the light. One or a plurality of wavelengths of light may be delivered by the plurality of fibers, or light emitting diodes (LEDs) may be used to provide one or more wavelengths of light. Electrical wires may be coupled to the LEDs and the wires may run along the barrier to a power source.

The plurality of optical fibers may be disposed on the outer surface of the elongate bag, or on the inner surface of the elongate bag, or in the wall of the elongate bag. The surgical drape may further comprise a distal ring (also referred to herein as a frame) coupled to a distal portion of the bag, and the plurality of optical fibers may be coupled to the distal ring or frame. The distal ring may comprise one or more lenses. The distal ring may be a closed annulus, or it may be an open ring, and the ring may take any shape such as a circle, ellipse, square, rectangle, polygon, etc. The drape may further comprise an optical window that is coupled to and disposed in a center portion of the distal ring. The optical window or the distal ring may include one or more optical waveguides. The surgical drape may comprise an optical window that is disposed adjacent a distal end of the elongate bag. The optical window may comprise a lens for focusing light passing therethrough. The optical window may comprise a central region with desired optical properties, and the plurality of optical fibers may be disposed circumferentially around the central region. LEDs may be coupled to the window to provide light instead of or in combination with the optical fibers. Whether optical fibers or LED, or both, one or more wavelengths of light may be provided. For example, white light may be provided to help a surgeon view the surgical field, or infrared or another wavelength may be provided to fluoresce a dye or other fluorescent agent that is disposed in the surgical field.

In another aspect, a system for protecting a surgical instrument comprises the surgical drape described previously and a camera disposed in the cavity defined by the inner surface of the elongate bag. The surgical instrument may comprise the camera. Any barrier herein may deliver on field lighting and may have one or more tubes or lumens that may be used for smoke evacuation during electrosurgery. The tubes or lumens may be coupled to an inner surface or an outer surface of the drape, bag or cover, or the tubes or lumens may be disposed in a wall of the drape or bag. Any barrier herein may also have one or more tubes or lumens for delivering a fluid to the surgical field, such as an irrigation fluid like saline, and they may be positioned similarly as the smoke evacuation tubes or lumens.

The system may further comprise an external light source that is optically coupled with the plurality of optical fibers. The light source may be an external light box such as a xenon lamp, or a laser, or LEDs. Any combination thereof is also contemplated.

In still another aspect, a method for illuminating a surgical field comprises providing a camera, disposing an elongate bag around at least a portion of the camera, illuminating the surgical field with light provided by optical fibers coupled to the elongate bag, and imaging the surgical field with the camera.

Illuminating the surgical field may comprise illuminating a dye or other compound in the surgical field and causing the dye or other compound to fluoresce. Imaging may comprise capturing the fluorescence. Illuminating may comprise delivering the light coaxially or non-coaxially relative to an axis of the camera.

In yet another aspect, a surgical drape for covering a surgical instrument comprises an elongate bag having an inner surface, an outer surface, and a wall therebetween. The inner surface defines a cavity and the cavity is configured to receive the surgical instrument. A distal ring is coupled to a distal portion of the elongate bag and at least one lighting element is coupled to the distal ring. The at least one lighting element is configured to emit light therefrom. A power source may also be disposed in or adjacent the distal ring. The power source is operably coupled to the at least one lighting element and provides power thereto.

In another aspect, a system for covering a surgical device comprises a barrier having a proximal portion, a distal portion, an inner surface, an outer surface, and a wall disposed between the inner surface and the outer surface, wherein the inner surface defines a cavity. The system also comprises a window disposed adjacent the distal end of the barrier, an illumination element disposed adjacent the window, and a surgical device disposed in the cavity, wherein the barrier provides a barrier between the surgical device and a surgical field and prevents contamination of the surgical field by the surgical device.

The barrier may comprise a bag, a drape, a pliable bag or drape, a cover, or a rigid housing. The barrier may be sterile or non-sterile.

The window may comprise an optical window, and the window may be coupled to an annular or other shaped frame coupled to the barrier. The illumination element may be coupled to the window. The illumination element may comprise one or more LEDs, a fiber optic cable, or a waveguide. The illumination element may deliver one or more wavelengths of light or speckled light to the surgical field.

The system may further comprise an optical element coupled to or disposed adjacent the window. The optical element may be coupled to or positioned adjacent an inner surface or an outer surface of the window, or the optical element may be embedded in the window. The optical element may comprise a lens, a polarizing element, a grating element, a filter element, a coating, a cladding, or a collimator element. The system may further comprise one or more service lines coupled to the window or the illumination element. The one or more service lines may comprise a fiber optic cable, a power line, a vacuum line, or an irrigation line. Thus, in some embodiments, a fiber optic cable may serve as both the illumination element and a service line. The one or more service lines may be coupled to the inner surface or the outer surface of the barrier, or the one or more service lines may be disposed in the wall of the barrier, and the one or more service lines may extend towards the proximal end of the barrier or housing.

The surgical device may comprise a camera, a microscope, an imaging element, or a sensor. The system may further comprise a coupling element for coupling the surgical instrument with the window.

In yet another aspect, a method for providing a barrier in a surgical field comprises providing a barrier having a cavity disposed therein, disposing a device in the cavity thereby forming a barrier between the device and the surgical field so as to prevent contamination of, or a breach of sterility in the surgical field by the device and illuminating the surgical field with light or other energy from an illumination element coupled to the barrier, wherein the light or other energy passes through a window or frame disposed in the barrier.

The method may further comprise coupling the device with the window or frame. Illuminating may comprise illuminating the surgical field with one or more wavelengths of light or speckled light from one or more LEDs, a laser, or an external light source. The one or more wavelengths of light or speckled light may pass through an optical element adjacent the window. The method may further comprise delivering light to the illumination element, providing power to the illumination element, irrigating the surgical field, or evacuating smoke from the surgical field with one or more service lines coupled to the barrier. The device may comprise a camera, and the method may further comprise imaging the surgical field with the camera through the window.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates localized illumination of a surgical field.

FIG. 2 illustrates general illumination of a surgical field.

FIG. 3 illustrates a cover disposed over surgical instrument.

FIGS. 4A-4D illustrate various embodiments of a surgical cover.

FIGS. 5A-5B illustrate exemplary embodiments of an optical window for use in a surgical cover.

FIG. 6 illustrates exemplary use of a cover disposed over a surgical instrument.

FIGS. 7A-7C illustrate exemplary embodiments of a cover.

FIG. 8 illustrates a schematic diagram of a barrier.

FIG. 9 illustrates an exemplary embodiment of a barrier with a handle.

FIGS. 10A, 10B1-10B2, and 10C illustrate another exemplary embodiment of a barrier with a handle.

FIGS. 11A-11B illustrate an illuminated barrier.

FIG. 12 illustrates another exemplary embodiment of an illuminated barrier.

FIG. 13 illustrates another exemplary embodiment of an illuminated barrier.

### DETAILED DESCRIPTION OF THE INVENTION

Specific embodiments of the disclosed device, delivery system, and method will now be described with reference to the drawings. Nothing in this detailed description is intended to imply that any particular component, feature, or step is essential to the invention.

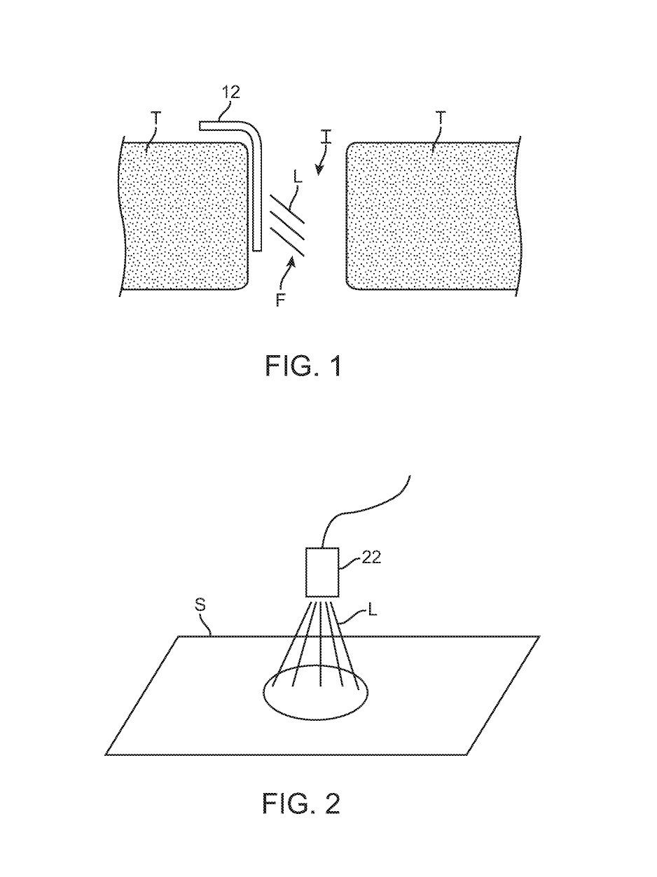

The present invention will be described in relation to an illuminated surgical drape (also referred to as a cover or barrier) that may be disposed over a camera, other piece of surgical equipment, a tool, or any other device or instrument. However, one of skill in the art will appreciate that this is not intended to be limiting and the devices and methods disclosed herein may be used in other surgical procedures with other surgical equipment, and also in other non-surgical procedures and with non-surgical equipment. FIG. 1 shows localized illumination of a surgical field F. In this situation, an illuminated surgical instrument such as an illuminated retractor 12 is disposed through an incision I in tissue T. An illumination element (not shown) such as fiber optics, a waveguide, or other lighting element coupled to the surgical instrument delivers light L and illuminates the surgical field F. Localized lighting is useful for illuminating smaller areas that require more intense light for visualization of the surgical field. All instruments used in the surgical field must be thoroughly cleaned and sterilized before use.

FIG. 2 illustrates illumination of a broader area. A light source 22 may be disposed above or otherwise adjacent a work surface S such as tissue or other anatomical parts of a patient. Light L is emitted from the light source 22 and a wider more diffuse beam of light illuminates the work surface. The light source may not be required to be sterile if it is outside of the surgical field, but it must be cleaned to prevent contaminants from falling into the surgical field. In still other optional variations, multiple fiber bundles are used, with each bundle delivering a different type of light preferably through an optical element like a lens. This allows some bundles to deliver a wide light output while others deliver a more narrow light output. Thus the user can control light output. As another example, this allows diffuse light to be delivered along with focused light. In some embodiments, the light source 22 is coupled to a surgical instrument such as a camera for imaging the illuminated work surface S. An example of this includes illuminating the work surface with a specific wavelength of light to cause dyes or other compounds to fluoresce on the work surface. The camera is able to capture the fluorescence and help a surgeon or other operator observe the fluorescence. This however, is not intended to be limiting, and one or more wavelengths of light may be delivered to the target such as a surgical field. Optionally, near infrared light may be delivered to tissue and the camera is used to image the tissue along with image processing in order to differentiate the illuminated tissue. More than one wavelength of light may be emitted from a single light emitter. In most surgical settings, it would be advantageous to cover the light source in order to prevent the light source 22 and any other surgical instrument coupled thereto from dropping dirt or other contaminants into the surgical field. Additionally, covering the light source prevents the surgeon or other operator from accidently touching the unsterilized light source and any adjacent surgical instrument, thereby minimizing the possibility of breaking the sterile field. Therefore reusable instruments may be covered by the barrier. Even if light is passed through beam splitters and exits the lens of a camera or other optical device (e.g. a surgical microscope), it still is advantageous to cover the microscope with a drape to prevent contamination.

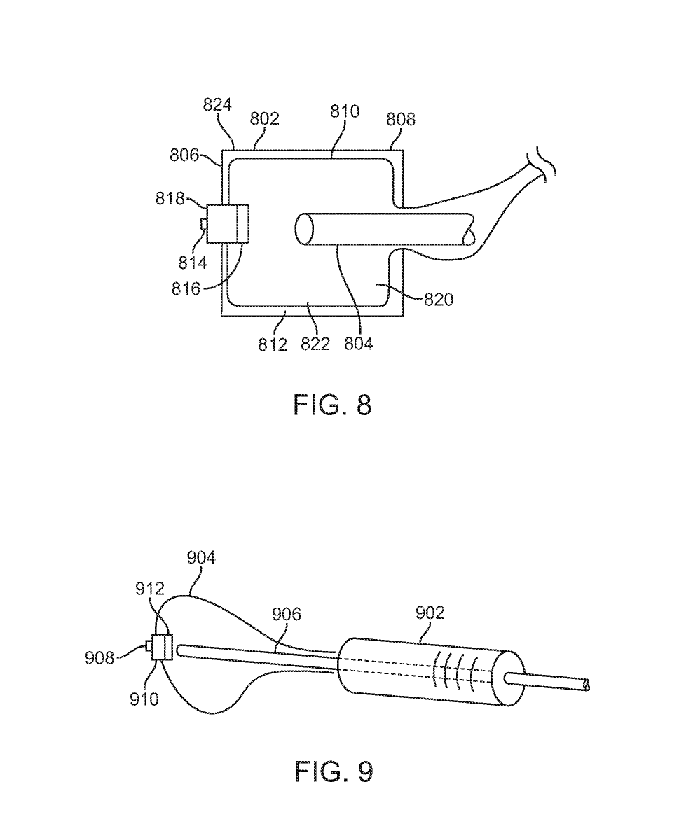

Thus, FIG. 8 illustrates a schematic diagram of a sterile barrier 802 which may be any of the exemplary embodiments disclosed herein. The sterile barrier 802 has a proximal portion 808, a distal portion 806, an inner wall surface 822, an outer wall surface 824, and a wall therebetween. The inner wall surface 822 forms a cavity or space 820. A surgical instrument, a device, a tool, or any other instrument 804 may be disposed in the cavity 820 thereby covering at least a portion of the device. When the barrier 802 is provided sterilized, the barrier thereby forms a sterile barrier between the instrument and the sterile field. Optionally, in some embodiments the barrier may not be fully closed at the proximal or distal ends and therefore the device does not provide a full barrier but acts only as a cover. The barrier in this or any embodiment may be fabricated from polymer such as a plastic bag, or a fabric, or any other material known in the art. Thus the distal portion of the instrument 804 may be positioned in or adjacent the sterile filed without fear of breaking the sterile field, or contaminating the sterile field with contaminants that may fall off the instrument. This allows any instrument such as a reusable instrument or piece of equipment like a camera, microscope or other device to be used during medical procedures without having to sterilize and completely clean the instrument. The proximal portion of the sterile barrier may have an opening to allow the instrument or device to be easily inserted and removed from the barrier. The opening may be tightly or loosely sealed against the instrument as required, using any number of techniques such as with tape, wire, cable ties, etc. The distal portion of the barrier may include a frame with a window 818 such as an optical window disposed in the frame so that light may pass therethrough without distortion. An optical element 814 may optionally be coupled to the window 818 in order to enhance the optical properties of the light passing through the window. The optical element may be disposed on either side of the window. In this or any embodiment disclosed herein, the window may also act as an optical element such as filter, polarizer, or lens, etc. Also, an illumination element 816 may also be coupled to the frame or adjacent the window in order to provide light that passes through the window and optionally through the optical element. One or more service lines 810, 812 may be coupled to the barrier and they may provide power, light, suction, vacuum, irrigation fluid, etc. to the distal portion of the barrier. For example, one service line may bring power to the illumination element so that the illumination element can deliver light. Another service line may provide suction to the distal portion of the barrier so that fumes may be evacuated from the surgical field during electrosurgery. A service line may be a fiber optic cable or bundle that delivers light from an external light source to the illumination element which may be a waveguide. Any combination of service lines is contemplated. The service lines may run along the inner surface of the barrier, along the outer surface of the barrier, or the service lines may be disposed within the wall of the barrier. The service lines preferably extend out of the proximal portion of the barrier so their free ends may be coupled to the corresponding service such as a vacuum, power, irrigation fluid, external light source, etc.

The barrier may be a thin flexible barrier such as a pliable membrane like a drape or cover including any of those disclosed herein, or it may be a more rigid housing such as an injection molded clam shell for holding the instrument or device. The instrument or device or tool may be any of those described herein. The illumination element may be any of the illumination elements disclosed herein. The optical element may be any of the optical elements disclosed herein. The window may be any of those described herein. Any combination of these features may be used with the barrier.

Examples of illumination elements include waveguides used to deliver light from either an external light source such as a light box or a laser, or a light source such as LED lights may be disposed adjacent the illumination element. The optical fibers delivering light may also be the illumination element.

Examples of instruments or devices or tools include but are not limited to cameras, sensors, microscopes, arms for holding retractors, etc. any of which may be sterile or non-sterile, single-use or reusable.

Examples of optical elements may include lenses, polarizing elements, gratings, filters, collimators, coatings, claddings, etc.

Examples of illumination elements may include lasers, fiber optic cables, LEDs, external light sources such as a light box like a xenon lamp, a waveguide, etc.

In the exemplary embodiment of FIG. 8, the instrument 804 is shown with its distal end remaining free of the window 818, optical element 814 or illumination element 816. However, optionally, in any embodiment described herein, the instrument 804 may be coupled to any of the window 818, optical element 814, or illumination element 816 with an engagement element. The engagement element may include magnets, fasteners, snap fits, press fits, other mechanical coupling mechanisms known in the art, or any other coupling means known in the art. For example, the window 818 may include a metal rim or frame around the window, and the instrument 804 may have one or more magnets adjacent its distal end which can then magnetically couple with the window.

In any of the embodiments, the illumination element whether a fiber optic that delivers light, LEDs, or a waveguide that delivers light, the illumination element, or the optical element may have a pattern that may be used to project an image onto the target surface. Projection of a pattern allows image analysis such as three dimensional reconstruction, depth measurements or spatial frequency domain imaging. The pattern may be projected using a particular specified frequency or polarization, and projection of the pattern may be turned off when the specified frequency or polarization is not used.

FIG. 9 illustrates an exemplary embodiment of a sterile barrier 904 that is deployed from a handle 902 and allows a user to easily insert a reusable or non-sterile instrument or device 906 into the barrier from the proximal end of the handle. The barrier may include any of the illumination elements 912, windows 910, or optical elements 908 disclosed herein. Any of the service lines disclosed herein may be employed in this exemplary embodiment.

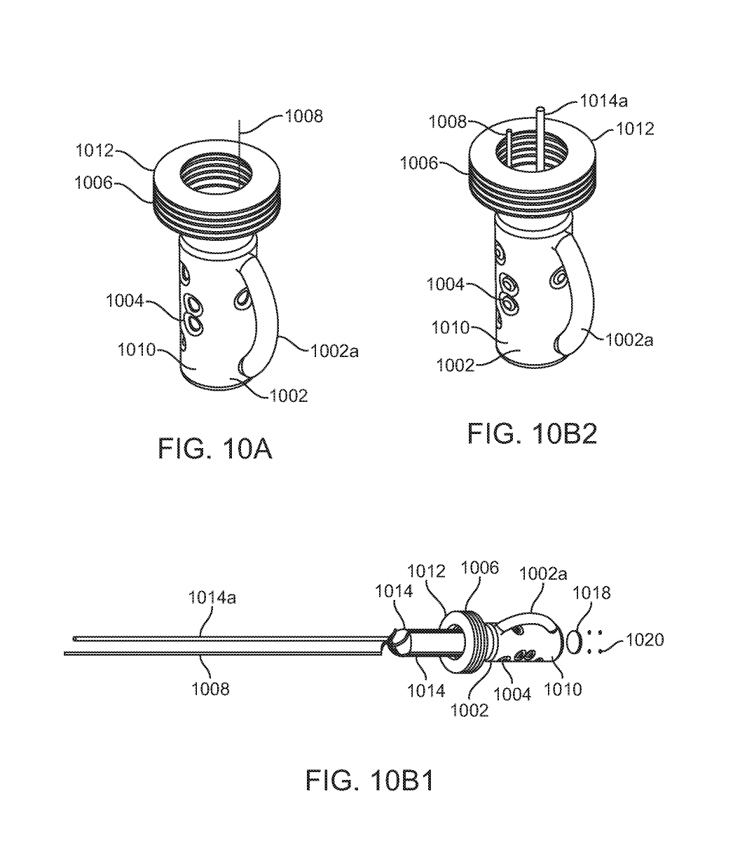

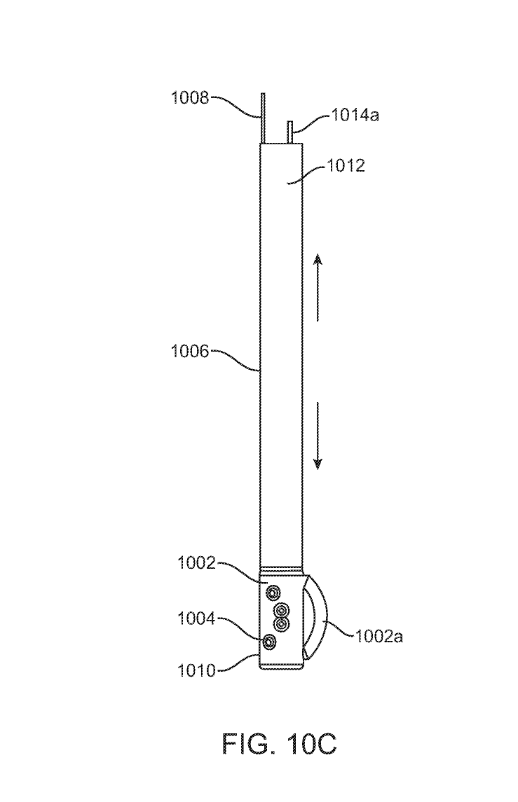

FIGS. 10A, 10B1-10B2, and 10C illustrate another exemplary embodiment of a sterile barrier with a hand piece. Hand piece 1002 includes a rigid housing which may be any shaped, but here is cylindrically shaped with a central channel and the hand piece is disposed on the distal 1010 portion of the barrier. An optional handle 1002a or hand grip may be included on the hand piece. A barrier, here a collapsed bag 1006 is coupled to the hand piece and a distal portion 1012 of the barrier is axially expandable from a collapsed or folded configuration to an expanded or unfolded configuration. Optional buttons 1004 or other actuation mechanisms maybe disposed on the handle and allow the operator to control the instrument that is disposed in the barrier and handle. For example, in the case of a camera, the buttons allow the operator to turn the camera on and off, zoom in/out, record, etc. An illumination element 1008 such as optical fibers run along the length of the barrier and into the handle to provide light to the target area such as a surgical field. The optical fibers may run through a central channel in the barrier and the handle, or the optical fibers may run along the inner surface or outer surface of the barrier. Or, the optical fibers may run within the wall of the barrier and handle. The distal portion of the handle may include any of the frames, optical windows, illumination elements, or optical elements disclosed in this specification.

FIG. 10B1 illustrates a medical instrument, here a camera 1014 inserted into the barrier 1006 and into the hand piece 1002. The distal portion of the camera (not shown) may be coupled to the distal portion of the hand piece, using any of the engagement mechanisms described in this specification or known in the art. The illumination element 1008 here includes one or more optical fibers extending proximally away from the hand piece. One or more service lines (e.g. power, data, etc.) 1014a extend proximally away from the camera. Optional optical elements 1020 and a window 1018 may be included in any of the configurations described in any of the embodiments disclosed herein. FIG. 10B2 shows the camera 1014 advanced fully distally until it is disposed in the hand piece and optionally coupled to the distal portion of the hand piece or window or frame.

In FIG. 10C, the barrier is retracted proximally (or the handle is advanced distally) to expand the barrier while the enclosed camera is moved into the sterile field while the proximal ends of the camera service lines (e.g. power and data) and the proximal end of the optical fibers are extended outside the surgical field where they may be coupled to the sources of power, a computer, a light source, etc. Thus, the camera may be used to image the target area and it is covered with a sterile barrier to prevent contamination in the sterile field. The barrier also provides similar protection to all of the cables and service lines. Optionally, in any of the embodiments, a cooperating element may be included in the barrier device that allows it to work uniquely with an instrument or device. This may be an electronic component such as a chip or other identifier that only permits the instrument or device to work with the barrier, or any other element that allows to two components to be operatively coupled together. Another example of this includes the use of an optical element adjacent the window in the barrier. A camera may require that optical element in order to provide quality imaging and without the optical element, the image would not be acceptable. This ensures that only a specific camera is used with the barrier and prevents third party devices from being used due to the required cooperation of the camera or other instrument with the corresponding cooperation element on the barrier, here an optical element.

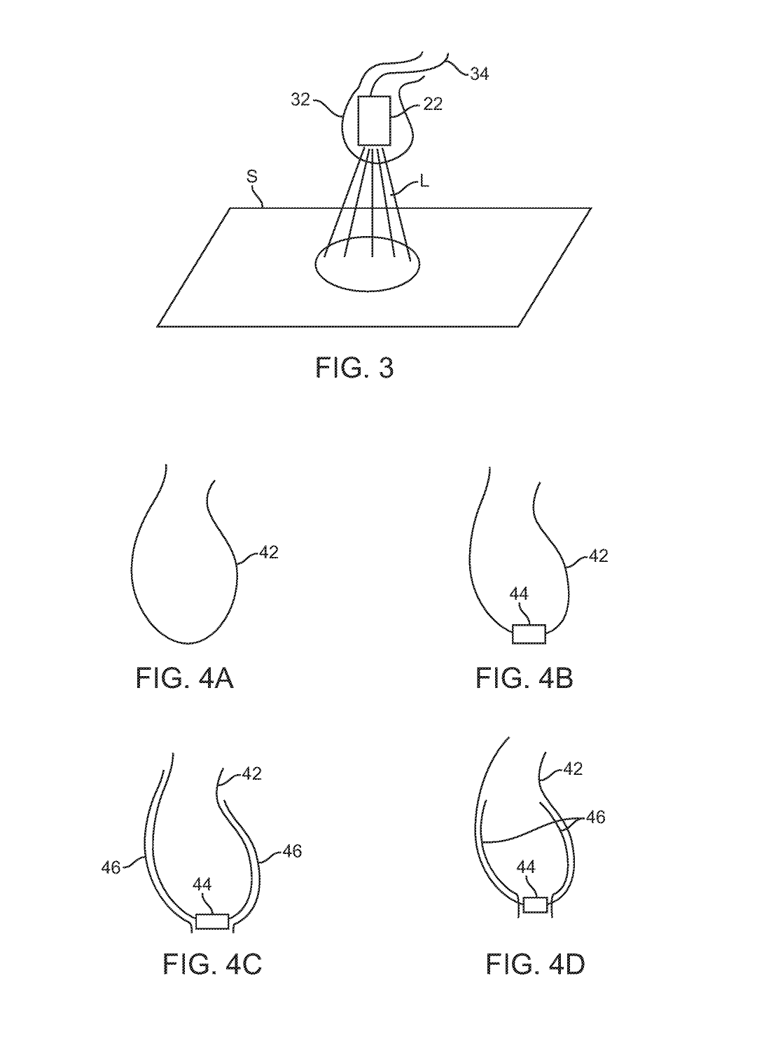

FIG. 3 illustrates a cover or barrier 32 that may be used to prevent contamination of the surgical field S by the light source 22 and any adjacent surgical instruments. The cover 32 may be a sheet of material or an enclosed bag that is disposed over the light source 22. It is preferably clear so that light L is emitted without significantly affecting the optical properties of the light. The cover 32 may be disposed over only a portion of the light source 22 or over the entire body of the light source 22. The proximal end of the cover may be taped or otherwise closed, or may include a drawstring for closing the open end of the bag. Additionally, the cover may also cover adjacent portions of any cables (e.g. power cables, fiber optic cables, or other service lines) 34 that may be coupled to the light source or adjacent surgical equipment. The cover is preferably a clear polymer such as polyethylene since this material is lightweight, and inexpensive. The cover may be supplied clean and sterile. However, in some circumstances, the cover may change the optical properties of the light or it may be desirable to provide the light without passing through the bag or by passing the light through a window in the distal portion of the bag that does not negatively affect the light.

FIGS. 4A-4D illustrate various exemplary embodiments of a cover that may be disposed over a light source and/or surgical instrument. In FIG. 4A, a cover is simply a plastic bag 42 that is disposed over the light source or surgical instrument. Because it may be undesirable to pass light directly through the cover, FIG. 4B illustrates the cover 42 with a frame having an optical window 44 in a distal portion of the cover. The optical window 44 is an area of the cover that has desirable optical properties and thus if the cover is disposed over a light source or a camera for example, the light provided by the light source will not be negatively affected by the optical properties of the window. Similarly, the window will allow a camera to image through the cover without resulting in image degradation. Or, in the case of a sensor, the sensor may sense through the window without interfering with the sensor's performance. The optical window may be a separate optical window that is coupled to the cover or it may be a distal portion of the cover which is been prepared to provide the desired optical properties. In this embodiment, or any embodiment disclosed herein, an optional frame is may be coupled to the cover and the widow may be disposed in the frame, or the window may be directly coupled to the cover without the frame. In FIG. 4C, optical fibers 46 are disposed on an outer surface of the cover 42. An optional optical window 44 is also included. The optical fibers may serve as illumination elements and may be used to provide light that illuminates the surgical field instead of light being supplied from within the cover and the cover may be disposed over a surgical instrument such as a camera. The fibers are coupled to an external light source such as a light box or laser which acts as the light source. FIG. 4D illustrates an alternative embodiment where the optical fibers are disposed on the inside of the cover and also an optional optical window 44 is also shown. In still other embodiments, the optical fibers may be embedded in the walls of the cover.

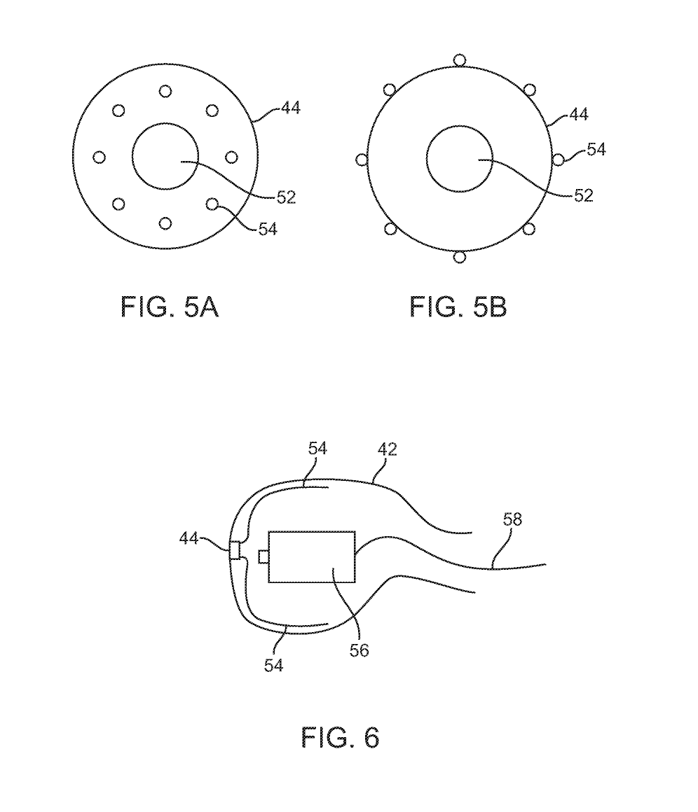

FIGS. 5A-5B illustrate exemplary embodiments of the frame and optical windows 44 which may be disposed at a distal portion of any of the barriers or the covers disclosed herein. In FIG. 5A the optical window includes an inner-most circular region 52 through which light is supplied or through which a camera views the work area, or through which a sensor senses, and this region has desired optical or sensor properties and may be formed from glass or a polymer having desired optical characteristics. For example, this region may simply be clear and have high transmission in order to pass light efficiently and to allow the camera to obtain a clear image. For sensors, the window has desired properties so that that sensor can sense through the window without interfering with the sensor signal. In other embodiments this region may have an optical element including any of those described herein, such as a lens to focus light or may be polarized or have other desired optical properties. Other optical coatings, diffusers, films or claddings may be disposed thereon in order to provide the desired optical properties. Optical fibers 54 are then disposed in the wall of the optical window to provide light. Thus the optical fibers are concentric with the inner most region 52 and the optical fibers and hence the light emitted therefrom would also be concentric with a camera's image axis. FIG. 5B is a similar embodiment to FIG. 5A with the major difference being that the optical fibers are disposed on the outer circumference of the window. The optical windows described in FIGS. 5A-5B may be used in any of the covers described in this specification. Any of these embodiments may include lenses or any of the optical elements disclosed herein. The lenses or optical elements may be coupled to the end of the optical fibers to shape the light. The lenses or optical elements may also be coupled to either side of the window including the inner surface or outer surface, or disposed between the window and the illumination element. The lenses or optical elements may be positionable, for example, so they may be toed inward or outward to focus or diffuse the light to a specific pattern at a specific distance from the camera or instrument.

FIG. 6 illustrates exemplary usage of the cover with a surgical instrument or device. The cover, here a polyethylene clear plastic bag 42 is disposed over a camera 56. The cover includes a frame coupled to the bag with an optical window 44 in the frame and near the distal end of the bag and through which the camera views the surgical field. The optical window may be any of the optical windows described in this specification. Optical fibers 54 bring light to the optical window and illuminate the field. The optical fibers may be on the inside of the cover as illustrated, or they may be on the outside of the cover, or integrated into the wall of the cover. The cover preferably covers the camera and at least a portion of the power and data cable or other service lines coupled thereto. Thus, in this exemplary embodiment, the lighting is concentric with the optical axis of the camera. The camera may optionally be releasably coupled with the frame or window using any of the coupling elements described in this specification or otherwise known in the art.

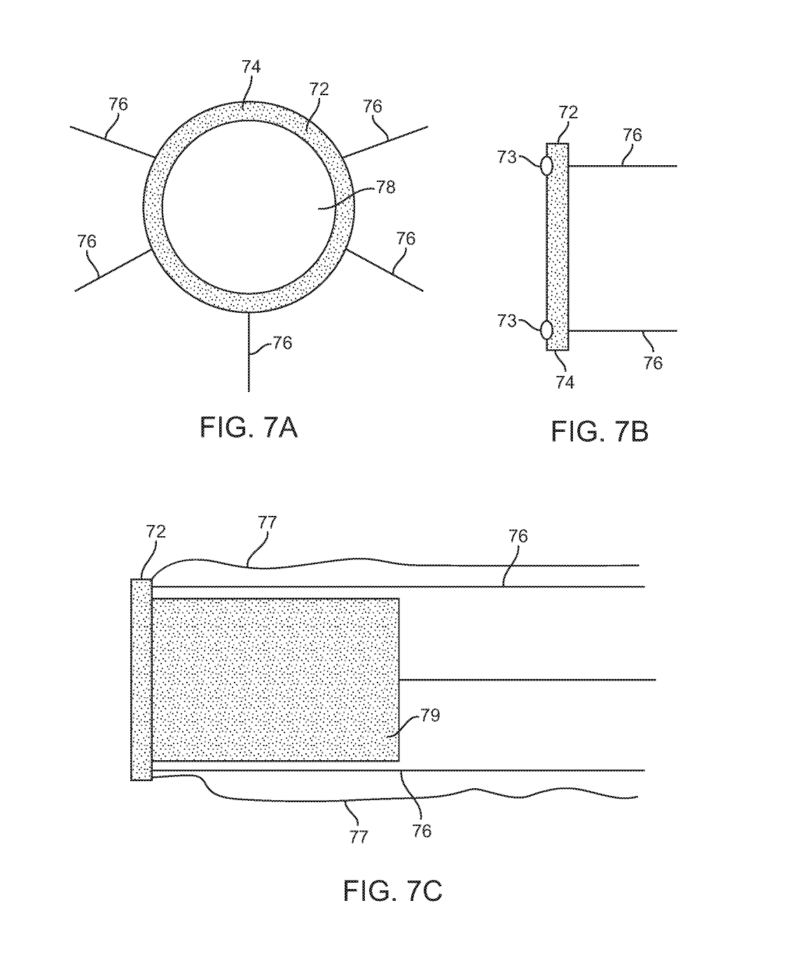

FIG. 7A illustrates an exemplary embodiment of a cover. A distal ring 72 includes an optional annular frame 74 to which a bag or other cover (not illustrated) may be coupled. Optical fibers 76 are coupled to the frame such that light from the optical fibers is emitted from a front face and distally of the annular frame 74. An optical window 78 is disposed in a center portion of the ring 72 and allows a camera or other instrument to have an unobstructed view of the target area such as a surgical field, and without distorting the image. In an alternative embodiment the distal ring does not have the annular frame and the optical fibers are coupled directly to the optical window.

FIG. 7B illustrates a side view of FIG. 7A showing the optical fibers coupled with the frame and passing therethrough so that light is emitted from the frame in a distal direction. Optional lenses 73 may be disposed circumferentially around the frame to shape and control the light. A bag or cover may be coupled to the distal ring 72. The optical fibers may be disposed in channels in the frame and this along with the optional lenses helps collimate the emitted light. FIG. 7C illustrates exemplary use of the cover seen in FIGS. 7A-7B. The distal ring or frame 72 may take the form of any of the distal rings or frames described herein. A cover or bag, also sometimes referred to as a drape 77 is coupled to the distal ring and may be used to cover a surgical instrument, here a camera 79 that is disposed in the cavity formed by the cover. Optical fibers 76 in this embodiment run along the outside surface of the bag and are coupled to the ring to deliver light distally through the frame or window. Alternatively, the fibers may run along the inside of the bag, or the fibers may be disposed within a wall of the bag. Proper illumination is then provided to allow the camera to image the target area through an optical window in the ring. Any single wavelength or multiple wavelengths of light may be delivered by this device.

In any of the exemplary embodiments, the optical fibers may be optically coupled to any number of light sources. For example the optical fibers may be coupled to a light source that allows the user to select the wavelength or wavelengths of light that are delivered to the surgical field. Thus, one or more frequencies of light may be supplied. In still other embodiments, light emitting diodes (LEDs) may be the source of the light that is transmitted over the optical fibers, or the LEDs may be coupled to the distal portion of the cover or the optical window or the frame to provide light to the surgical field directly without requiring the optical fibers.

As previously mentioned, the light supplied by the optical fibers may be or any other illumination element (e.g. LEDs or external source) used to excite fluorescent dyes in the surgical field, and the camera may be used to help visualize the fluorescence. Fluorescent dyes and other compounds may be used which bind to specific tissue thereby allowing a surgeon to precisely identify the location of target tissues and distinguish them from non-target tissue. Optionally, in any of the embodiments disclosed herein, the optical fibers may be used to deliver speckled, de-speckled or both speckled and de-speckled light to the target. Optionally, in any of the embodiments disclosed herein, light elements may be disposed adjacent the light emitting end of the cover, and the light elements may provide speckled, de-speckled, or both speckled and de-speckled light. Speckled light may be provided by a laser and may be advantageous for certain imaging such as when used with an imaging source such as a camera to image blood flow in vascular structures. This may be referred to as speckle contrast imaging and allows mapping and measurement of blood flow distribution in vessels. Thus, in any of the embodiments disclosed herein, some fibers may be optically coupled with diffuser lenses to provide the desired light while other fibers may not have lenses.

In any of the embodiments, lenslets may be used. Any of the waveguides or the lenslets may optionally comprise refractive, diffractive, or holographic structures. These structures may be used in conjunction with an appropriate wavelength of light to modify the light to have desired properties such as a specific pattern.

Any of the barriers or covers may be sterilized by methods known in the art, and they may be single use only or they may be reusable.

The sterile barriers have been discussed primarily with respect to covering a surgical instrument or device such as a camera. However, one of skill in the art will appreciate that any of the embodiments disclosed herein may be used to cover any instrument, device, tool, or work piece. For example, the barriers disclosed herein may be used to cover a surgical arm that is used to hold a surgical retractor. Thus a barrier with an illumination element may be used to deliver light to a waveguide coupled to the retractor, instead of light cables being coupled to the retractor. In this or any embodiment described herein, one or more sources of light may be used to deliver one or more types of illumination to the target via the optical fibers or by any other means, such as different wavelengths, different intensities, etc.

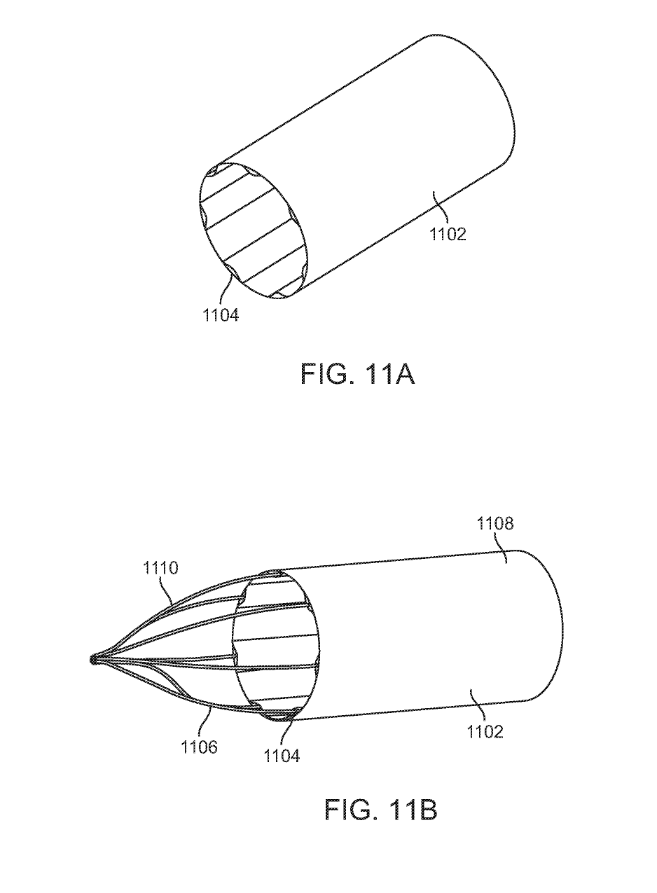

FIGS. 11A-11B illustrate an exemplary embodiment of an illuminated cover or partial barrier that may include any of the features of the other barriers disclosed herein. The cover or partial barrier 1102 is generally an elongate tube having an inner central channel into which a device, instrument or tool may be inserted into order to separate it from the surgical field and optionally provide a sterile barrier therebetween. The proximal and distal ends are preferably left open in order to allow instruments, cables, etc. to pass into or pass out of the cover or partial barrier. Optional channels 1104 are disposed either on the inner surface, outer surface, or in the wall of the barrier or cover. The channels allow optical fibers or other illumination elements to be disposed in the channels in order to provide light. FIG. 11B illustrates a plurality of optical fibers 1106 disposed in the channels 1104 so that light may exit the opposite end 1108 of the barrier or cover. The fibers extend from the other end 1110 away from the sterile field where they may be coupled to a light source (not shown). This embodiment may be used with any number of items in which it is desirable to cover them up with a barrier or cover. For example, articulated arms are often used in surgical procedures and they are attached to a patient's bed frame or a wall of the room and an instrument such as a retractor or other item is coupled to the arm. The illuminated barrier or cover may be used to provide a partially sterile barrier between the arm and the surgical instrument and prevent contamination of the sterile field, keeping in mind that the ends of the cover are open and thus a complete sterile barrier is not formed. The cover is preferably used for cable management.

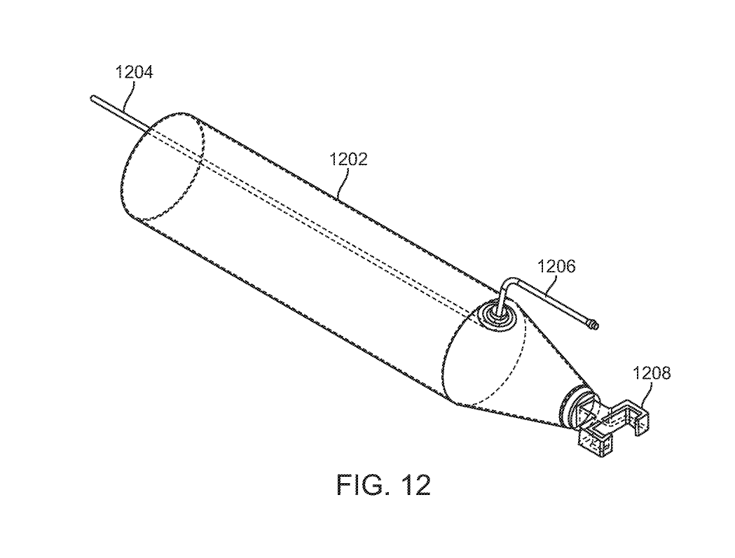

FIG. 12 illustrates another exemplary embodiment of an illuminated barrier which may incorporate any of the features of the other embodiments disclosed herein. The barrier 1202 has a central channel into which an instrument or device may be inserted to prevent it from contaminating the surgical field. One or more optical fibers 1204 may run along the length of the barrier to provide light to the surgical field. The distal end of the barrier may be closed off to prevent contamination at the distal end. An optional coupling mechanism 1208 may be disposed at the distal end and this may be used to couple the barrier to an instrument in the surgical field such as a retractor. The optical fibers 1206 may be coupled to an illumination element on the instrument.

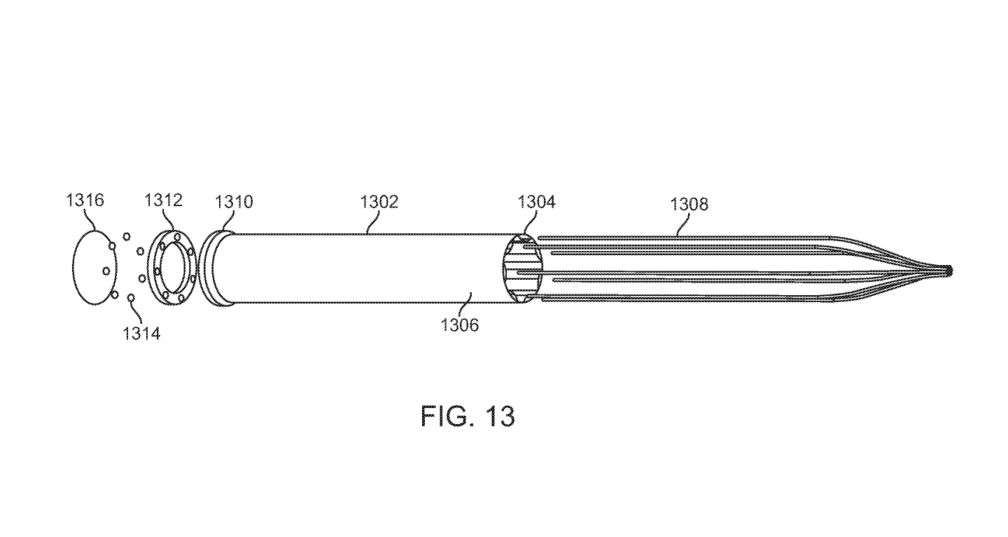

FIG. 13 illustrates another exemplary embodiment of an illuminated barrier 1302 is preferably an elongate and flexible bag having a central channel for receiving an instrument or device. Channels 1304 extend along the length of the barrier and optical fibers 1308 is disposed in the channels to deliver light to the distal end 1310 of the barrier. The optical fibers extend from the proximal end 1306 so they are away from the sterile field and they may be coupled to a light source (now shown). A frame 1312, here an annular ring is coupled to the distal end of the barrier to provide a rigid support and this allows other items to be coupled to the ring. For example, a camera or other instrument may be releasably coupled to the ring. Additionally, optionally the barrier may also include optical elements 132 and a window 1316 coupled to the ring. The optical elements may be disposed on either side of the optical window, and the optical elements, window, and ring may be any of those described in this specification.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-24. (canceled)

25. A surgical cover for covering a surgical instrument, said cover comprising:
an elongate bag having an inner surface, an outer surface, and a wall therebetween, the inner surface defining a cavity, the cavity configured to receive the surgical instrument; and
one or more illumination elements coupled to the elongate bag, the one or more illumination elements configured to deliver light to a distal end of the elongate bag, wherein the one or more illumination elements are disposed on the outer surface of the elongate bag, disposed on the inner surface of the elongate bag, or disposed in the wall of the elongate bag.

26.-28. (canceled)

29. The surgical cover of claim 25, wherein the one or more illumination elements comprise a plurality of optical fibers.

30. The surgical cover of claim 25, further comprising a distal ring coupled to a distal portion of the bag, and wherein the one or more illumination elements are coupled to the distal ring.

31. The surgical cover of claim 30, wherein the distal ring comprises one or more lenses.

32. The surgical cover of claim 30, further comprising an optical window disposed in a center portion of the distal ring.

33. The surgical cover of claim 32, wherein the optical window or the distal ring are optical waveguides.

34. The surgical cover of claim 25, further comprising an optical window disposed adjacent the distal end of the elongate bag.

35. The surgical cover of claim 34, wherein the optical window comprises a lens for focusing light passing therethrough.

36. The surgical cover of claim 34, wherein the optical window comprises a central region with desired optical properties, and wherein the one or more illumination elements are disposed circumferentially around the central region.

37. A system for protecting a surgical instrument, said system comprising:
the surgical cover of claim 25; and
wherein the surgical instrument comprises the a camera, and wherein the camera is disposed in the cavity.

38. The system of claim 37, further comprising an external light source optically coupled with the one or more illumination elements.

39.-44. (canceled)
